(19) United States
(12) Patent Application Publication
(10) Pub. No.: US 2011/0284013 A1
(43) Pub. Date: Nov. 24, 2011

(54) CHRONOCENTRIC METHOD FOR IMPROVING EFFECTIVENESS OF WEIGHT LOSS TECHNIQUES

(75) Inventor: Gregory Sadkhin, Staten Island, NY (US)

(73) Assignee: The New Level Sadkhin Group, Inc., Brooklyn, NY (US)

(21) Appl. No.: 13/112,673

(22) Filed: May 23, 2011

Related U.S. Application Data
(60) Provisional application No. 61/333,285, filed on May 21, 2010.

(51) Int. Cl. A61B 19/00

(52) U.S. Cl. 128/898

(57) ABSTRACT

The method of the present invention comprises various steps of employing at least one mechanical, chemical, biochemical, pharmaceutical, electrical, magnetic or other treatment, to a living organism (optionally targeting at least one organ or organ system thereof), said living organism being human or animal, to achieve at least one predetermined treatment goal (representative of a treatment profile), at least a portion of the steps comprising at least a portion of the following procedures: (1) conducting comprehensive multi-profile diagnostics and identifying at least one of a target organ, and/or a target organ system, of a target living organism, that requires at least one predetermined treatment (e.g., medical, preventative, fitness, etc.); (2) establishing at least one optimal recommended treatment type(s) in response to the at least one predetermined required treatment, for said identified target organs and/or organ systems; and/or (3) determining the chronosystem context of biological rhythms of the entire target organism, at least one of a target organ, and/or a target organ system, wherein advantageously, the inventive method greatly improves the effectiveness of at least one of the above-noted employed treatments and/or treatment sets, in their application individually and in conjunction with one another, by conducting the treatments cyclically, on the basis of a target chronosystem of the target organism, of at least one of the target organs, and/or of the target organ system (for example, varying the dosage, frequency, and/or periodicity of the treatment applications, and/or varying other treatment application parameters in accordance with matching, or otherwise correlating, the target chronosystem with at least one predetermined chonologic factors (seasons, month of a year, time of day, etc.).

### CROSS REFERENCE TO RELATED APPLICATIONS

The present patent application claims priority from the commonly assigned co-pending U.S. provisional patent application 61/333,285 entitled “Chronocentric Method for Improving Effectiveness of Weight Loss Techniques”, filed May 21, 2010.

### FIELD OF THE INVENTION

This invention relates to a method for reducing an individual's weight. More particularly, this invention relates to a method wherein biologically active hunger control points and/or areas on the skin are stimulated in accordance with a predefined configurable Chronocentric cyclical schedule to reduce the individual's food cravings, thereby resulting in weight loss.

### BACKGROUND OF THE INVENTION

The Chronosystem is the system that regulates the internal rhythm of the body's organ and organ systems. You can think of the Chronosystem as being composed of biological “clocks”. These biological clocks are responsible for maintaining the body's proper rhythms, and can be affected negatively or positively by the environment. There is a “big” biological clock located in the part of your brain called the hypothalamus that is in charge of maintaining the general balance of the body. There are also many “small” clocks which are most specialized to particular organs and functions. These “small” clocks may or may not communicate with the “big” clock, depending on what function it serves.

There are many different rhythms in the body. For example, if the internal rhythm of the heart gets interrupted, then that person will suffer from heart disease. Similarly, if one's lung rhythm gets interrupted, then that person is likely to suffer from asthma and other breathing related illnesses.

As a people age, their internal rhythms get offset with time. By regulation of this system using methods such as the Sadkhin Method, these rhythms get restored to a previously younger state, which result in better health, energy, and prolongs youth.

It is a scientific fact that calorie restriction with optimal nutrition (CRON) helps restore the body to a youthful state, prolonging life and fighting disease (we can provide you with studies to support this if asked). It is very difficult to follow a CRON plan without the aid of the Sadkhin Method, which introduces hunger control techniques to create a synergy that not only results in weight loss, but overall greater health—Bodies are like clocks, without proper maintenance, they get offset and do not function properly.

### BRIEF DESCRIPTION OF THE DRAWINGS

In the drawings, wherein like reference characters denote corresponding or similar elements throughout the various figures:

FIG. 1 is a diagram listing the major human organ systems influenced by the method of the present invention;

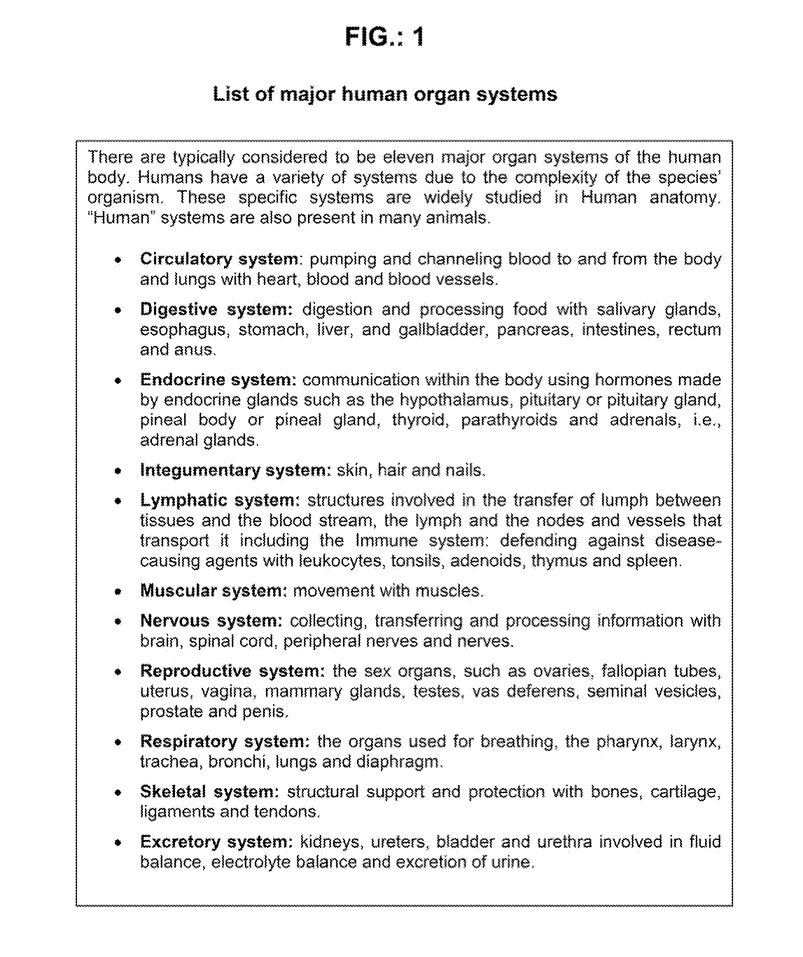

FIG. 2 is a diagram listing various exemplary negative chronizer triggers negatively impacting the major human organ systems influenced by the method of the present invention;

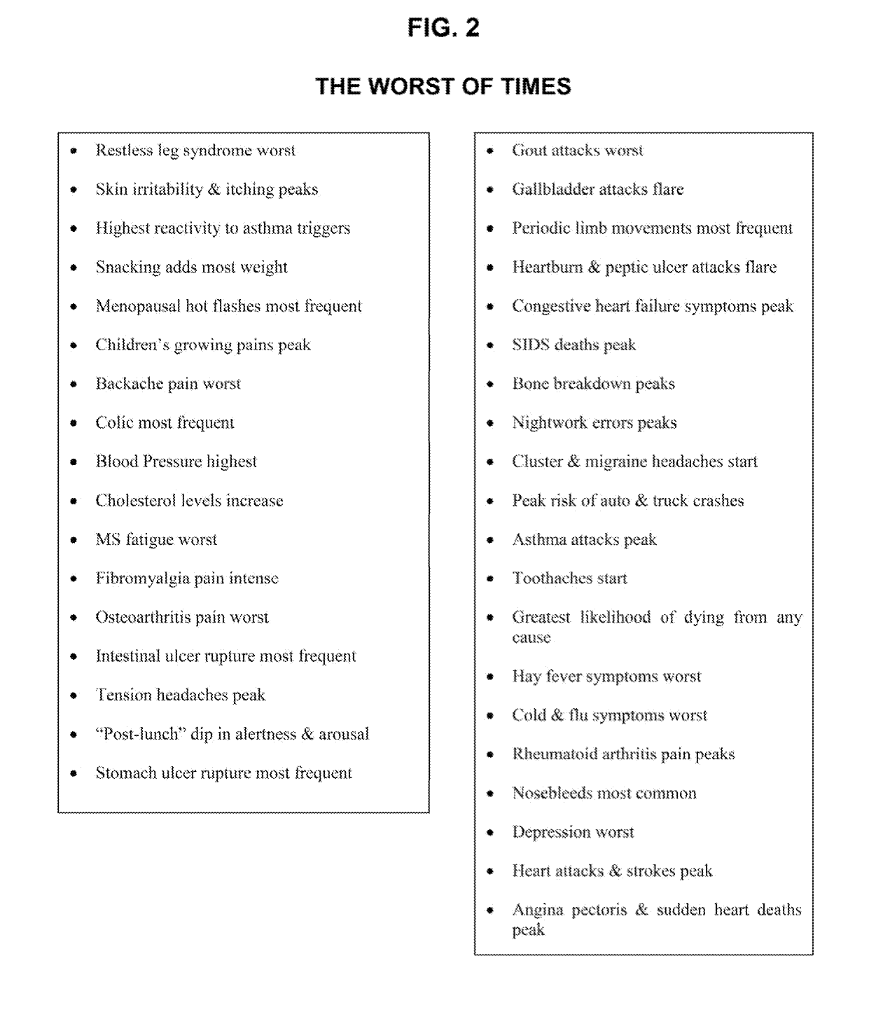

FIG. 3 is a diagram listing various exemplary chonocentric chronizer triggers affecting the major human organ systems influenced by the method of the present invention;

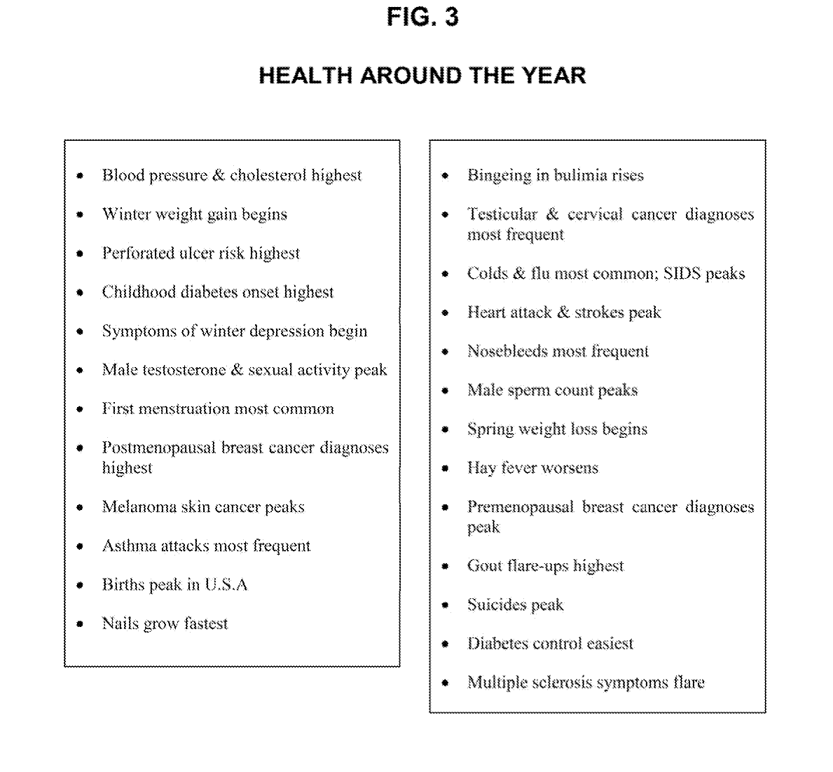

FIG. 4 is a diagram listing various exemplary positive chronizer triggers positively impacting the major human organ systems influenced by the method of the present invention; and

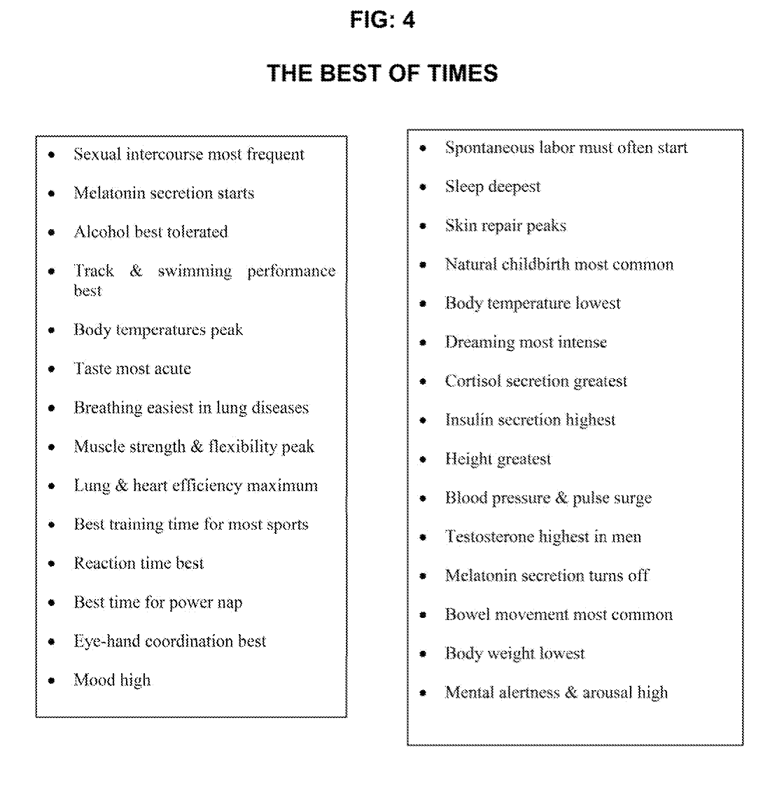

FIG. 5 is a schematic graph diagram showing an exemplary distribution of various values under a novel Sadkhin Complex Health Index determined in connection with utilization of the method of the present invention.

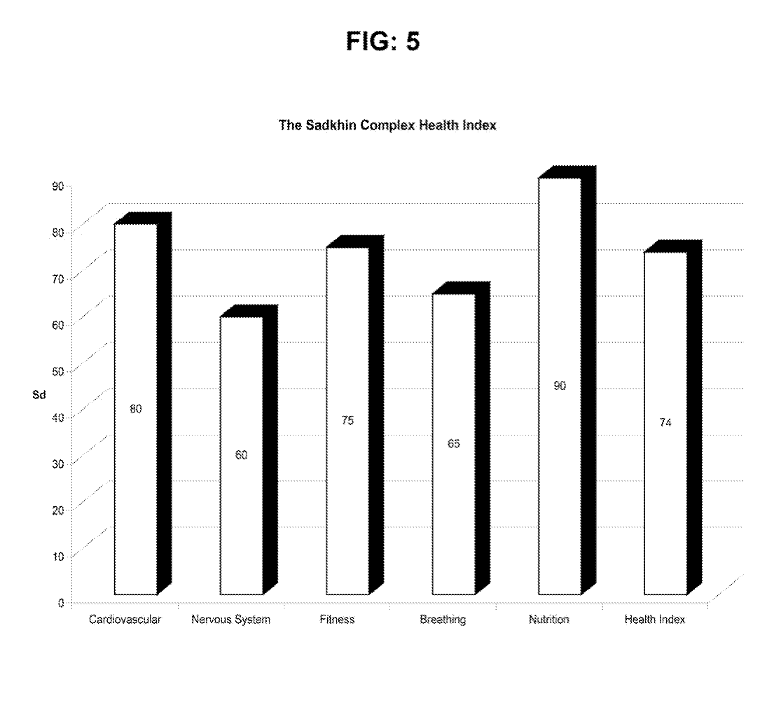

### DETAILED DESCRIPTION AND SUMMARY OF THE PREFERRED EMBODIMENTS

Discynchronization of biological rhythms and the absence of preventative measures causes obesity. Obesity is accelerated aging. Only when we consider these factors, can we take concrete steps to win the battle against obesity

In accordance with the present invention, the Sadkhin Complex achieves rapid and safe weight loss and deceleration of the aging process through the integration of multiple modalities such as the Sadkhin Method, restrictive feeding, physical activity and behavioral modification into a unified system using chrono-synchronization of several areas of the brain and peripheral oscillators. The process involved active stimulation of hunger and satiation centers by sending signals to all regulatory pathways that influence energy expenditure, appetite, nutritional fulfillment, and fat deposits.

The method of the present invention comprises various steps of employing at least one mechanical, chemical, biochemical, pharmaceutical, electrical, magnetic or other treatment, to a living organism (optionally targeting at least one organ or organ system thereof), said living organism being human or animal, to achieve at least one predetermined treatment goal (representative of a treatment profile), at least a portion of the steps comprising at least a portion of the following procedures:
(1) conducting comprehensive multi-profile diagnostics and identifying at least one of a target organ, and/or a target organ system, of a target living organism, that requires at least one predetermined treatment (e.g., medical, preventative, fitness, etc.);
(2) establishing at least one optimal recommended treatment type(s) in response to the at least one predetermined required treatment, for said identified target organs and/or organ systems; and/or
(3) determining the chronosystem context of biological rhythms of the entire target organism, at least one of a target organ, and/or a target organ system, wherein advantageously, the inventive method greatly improves the effectiveness of at least one of the above-noted employed treatments and/or treatment sets, in their application individually and in conjunction with one another, by conducting the treatments cyclically, on the basis of a target chronosystem of the target organism, of at least one of the target organs, and/or of the target organ system (for example, varying the dosage, frequency, and/or periodicity of the treatment applications, and/or varying other treatment application parameters in accordance with matching, or otherwise correlating, the target chronosystem with at least one predetermined chonologic factors (seasons, month of a year, time of day, etc.).

Circadian time-keeping appears to be a fundamental quality of life. In modern times, the issue of increased rates of adult and child obesity has been repeatedly brought up in the Unites States Senate.

Complications of Obesity:
Metabolic
Anatomic
Degenerative
Psychological
Neoplastic

In an attempt to better understand the proves of disease, we split the human body into twelve organ systems—each of these 12 systems represents a distinct part of the human body. The systems must work together to ensure that the one big system, the human body, works properly. Through extensive scientific research, Dr. Sadkhin was first to identify and introduce a new system, the Chronosystem, as one of the most important systems of the human body. The Chronosystem is the missing Chronos of the human body, uniting the 12 systems helps us better understand the human body.

The Chronosystem is represented by the anatomophysiological structure responsible for various functions of the human body. The science of chronobiology and chronomedicine is the study of the evidential rhythmicity and periodicity of the working organs and tissues of the human body and the functional processes that govern them.

Practices such as eating fresh, raw foods, drinking whole fat milk, and adjusting their menus and physical activity to the changing of the seasons all create a peak environment. Russian scientists discovered a group of time quants inside the human body. These time quants depend only on the speed of fermentative reaction, not on real time. We can use temperature to regulate the size of these time quants. We have gathered knowledge of chronomedicine to finally be able to organize the information and realize that proper operation of human organs depends on time. Based on this knowledge, we can prevent and treat obesity, the process of accelerated aging. One of the basis of chrnomedicine is time. Time rules our biological clock. External elements, such as the use of electricity to extend our walking hours and change sleep patterns alter our behavior. Understanding how our biological clock interacts with and reacts to the external environment enables us to improve the quality of life and promote longevity.

The National Institute on Aging is currently conducting extensive research on biological rhythms, including the effects of normal and disordered biological rhythmicity on the aging nervous system and cellular and molecular mechanisms controlling biological rhythmicity within the aging nervous system. This means that, essentially, if we can regulate the area of the brain which houses our biological clock, we can regulate our aging process. The Sadkhin Comples is a system that comprises of several components to cure obesity, increase the quality of life, and promote longevity.

Chronizers are the key to the Sadkhin Complex. A chronizer is an environmental agent or event that provides a stimulus which sets or resets the biological clock of an organism. For weight control, the Sadkhin Complex alters the activities of various enzymes that control fat metabolism, causing more fat to be burned for energy and less to be taken up into the body, decreasing the number and size of the fat cells and significantly decreasing total body weight. Edipose tissue is an active endocrine organ that secrets a number of hormones/cytokines including leptine, resistin, adiponectin, IL-6. And TNFa. This adiposite-derived proteins (adipocytokins/adiopokines) play critical roles in body energy homeostasis and metabolism. Chronizers are the keys to The Sadkhin Complex. The chronizer is used to control and regulate hunger and satiation. The human skin is a living organ of the body, just like other organs such as fat tissue. Skin has always played an important role in medicine. For example in ancient times, doctors looked at newborn's skin and birthmarks to try to predict the child's future. The skin contains a set of Biologically Active Points. Sadkhin Points are a type of Biologically Active Points.

Clinical Studies confirm the specificity of Sadkhin Points as compared to other points on the body. Stimulation of Sadkhin Points induces changes in the activity of the sensory and motor functions of the brain, as well as the autonomic nervous and endocrine systems, accompanied by changes in the electroencephalogram and electromyogram and increases in levels of certain amines, peptides, hormones and other physiologically active substances in certain structures of the brain and spinal cord and in the cerebrospinal fluid and blood. This leads to complex reflex responses, which take the form of development of sedation, analgesia, and changes in emotional reactions and psychological functions. Reflex responses are also manifest in the form of an antispasmolytic effect, return to baseline parameters of peripheral and central circulation, restoration of optimal functions of the digestive, respiratory, endocrine, immune, and muscles systems, and normalization of various metabolic processes.

Activation of Sadkhin Points leads to intensification of the body's own defense mechanisms in the struggle against functional illnesses and an increase in the functions of all the regulatory systems, enabling the body to produce an effective counterattack on the continually changing effects of multiple environmental factors.

Sadkhin Points are independent receptor organs, primarily reacting to changes in ambient electric and magnetic properties. Sadkhin Points can be activated only by the particular stimuli chosen by Dr. Sadkhin and appropriated to the somatosensory systems. The polymodality manifested by Sadkhin Points, as independent receptor organs, makes them similar to electroreceptors, which have shown not only high sensitivity to changes in electric fields, but also sensiticity to magnetic fields, mechanical, and thermal stimulation. The idea that Sadkhin Points may serve the function of electromagnetic receptors is further supported γ the similarity of the morphofunctional characteristics of these points (high concentrations of nerve ending, low electrical resistance, polymodality).

Morphological studies of the skin find a number of distinguishing features in Sadkhin Points areas, namely, thinner epidermis in looser dermal connective tissue, large number of free nerve endings of a cholinergic nature, as well as encapsulated endings in the form of Krause end bulbs, Meissner's corpuscles, Fater-Pacinian corpuscles, etc., nerve fibers and vessels surrounded by adrenergic plexuses, substantially innervated vessel networks, and lymphatic vessels.

Tissues containing these points feature increased concentration of mast cells generating biologically active substances, such as histamine, acetylcholine, and serotonin, that take part in the nerve and vessel regulation. It has been established that the synthesis of melanin, catecholamines, and a number of enzymes and vitamins occurs in the skin.

The way Sadkhin Points differ from adjacent skin areas and tissues is also defined by the unique bio-physiological parameters: lower resistance to both direct and alternation currents, higher value of electric potential, different temperature and infrared radiation, and other factors. Taken together, these observations point to a higher functional activity of the skin in areas of Sadkhin Points projection. Walls of nerve vessels contain adrenergic fibers plexuses and a large quantity of nerve endings classified as baro-, chemo-, and thermorceceptors.

By stimulating Sadkhin Points in a chrono-synchronized manner, we replace weak and long-term natural stimulation of the ecoceptic sensitivity system receptors with more intense, but shorter term artificial stimulation. This is sufficient to trigger the activity of neuroregulatory systems of the brain, which is the key component of the adaptive mechanism. The interaction of the Sadkhin Points with special metal balls, which are homeopathically potentiated materials, is the source of specific external electromagnetic signals.

Chrono stimulation, as first defined by Dr. Sadkhin, is marked by applying even pressure or any other modalities to steel balls attached to Sadkhin Points. The goal of this stimulation is to obtain a pharma-therapeutic effect. The ideal strength levels of multi-modality stimulation, at equal intervals, allow the body to reach a phase of stimulation and amplitude in which the body begins to use its own preventative and self-restoring forces.

During chrono stimulation by metal balls on Sadkhin Points, an electric impulse is achieved. This impulse affects the hypothalamus and the center of the satiation and hunger, as well as the central nervous system. This achieves a much better and faster result because the effects of the central nervous system on various organs are much more varied and complex. Directives to all organs go through the central nervous system because the system's electro conductivity is much higher that that of other systems. Because the electrophysiological properties of organs and tissues are restored during chrono stimulation on Sadkhin Points, the electrical charges we receive are so precise that they coincide with properties of specific organs.

To control hunger, we use five types of modality, both static and dynamic. Statically, we obtain a constant acupressure effect by attaching the ball to the skin with tape. Dynamically we get a reaction by chrono synchronization every 2 or 1½ hours, depending on the group of organs we are trying to affect and by chrono simulation at a special time such as 3 and 7 pm. This special time changes every 10 days.

After 10 days, the sensitivity to the signals, both static and dynamic, markedly lowers and we must affect a different group of organs to obtain a constant strong pharmacotheraputical effect on the hunger and satiation centers and other affected organs and groups of organs.

A study called “The Human circadian system operates as a phase locked loop” was conducted by Steven B. Lowen, Ph. D. and Martin H. Teicher, M.D., Ph.D., at the Developmental Biopsychiatry Research Program, McLean Hospital, and consolidated Development of Psychiatry at Harvard Medical School.

This study showed that the human circadian system bears a remarkable similarity to a well-studied circuit from electrical engineering: the phase locked loop. This similarity includes the ability to entrain to external chronizers, and the generation of free-running oscillations near the desired period in the absence of oscillators. The entrainment strength depends on both the period and amplitude of the chronizers. The dramatic connection between human circadian systems and phase locked loops is important for two reasons. First, extensive results of phase locked loops exists in electrical engineering literature, all directly applicable to the study of human circadian systems. Second, the structure of phase locked loops suggests avenues of research toward understanding the physiology of the human circadian system.

The five types of modality that may be advantageously utilized in accordance with the present invention are:
Accupressure—This is the stimulation of mechano receptors by means of applying pressure
Thermo Pressure—This is the process of Warming the Sadkhin Points during stimulation. Thermo pressure achieves deep heating and long effect. With this, comes a feeling of pleasant heat.
Galvano Pressure This is the process of obtaining a micro charge between the skin (which has a negative change) and metal (which has a positive charge). A chemical effect, which intensifies the treatment, can also be added. The addition of a chemical effect is called galvano pressure.
Electro pressure—This affects informative and regulation processes of the body. These processes are regulated through the nervous system, since the electrical current is a physiologically adequate regulator of the nervous system.
Magnetic pressure This is the local effect of constant or intermittent magnetic field with the goal of healing. The effectiveness of magnetic pressure depends on sedative, painkilling, anti-inflammatory, and regenerative properties, activating homeostasis, electromagnetic balance, and immunoreactive reaction.

The patient's position during chrono stimulation is crucial. To maximize the effect of chrono stimulation on the Sadkhin Points, the patient should be seated, with the head bent down (this is known as the fetal position). Dr. Sadkhin tested the effects of chrono stimulation on Sadkhin Points both before and after treatment. The tests were conducted on clients sitting down with the head bent down, sitting down with the head held up, and standing up with the head held upright. The tests show that the effect of chrono stimulation is optimal in the sitting position with the head bent down, using slow stimulation.

In addition to discovering and developing Sadkhin Points, Dr. Sadkhin has invented a method and apparatus for use in treating Biologically Active Points on a patient's skin. Currently, Dr. Sadkhin is working on early diagnostics of organs and organ system with unique precision. The other part of this invention is fact lifting without surgery which works by measuring biologically active points of the fact connected with aging and influences diagnosing problem areas on the fact and the influence of multiple modality to obtain desirable therapeutical effect.

Dr. Sadkhin has another patent for a therapeutic and diagnostic method and apparatus. This unique treatment comprises selecting a zone of a patient's iris to be treated, monitoring the eye to determine a position of the selected zone and in accordance with the determined position of the one, directing energy of the predetermines type and magnitude toward the zone.

Dr. Sadkhin conducted extensive population studies with his clients. The 448 clients in the study lost weight rapidly (an average of 18 pounds a month), while their energy levels increased. In addition, clients started feeling and looking younger. These studies led to the development of the Sadkhin Complex.

How “chronoplan” got its name: The acronym for Calorie Restriction with Optimal Nutrition is “CRON”. This CRON plan is a major part of the Sadkhin Method. “Chronos” is the Greek god of time. This control of time is an integral part of the Sadkhin Method which seeks not only to help people lose weight, but also to prolong their life. Combining these two words, “chronoplan” embodies the purpose and function of the Sadkhin Complex.

The tsubo balls (steel spheres), which is the tool that Dr. Sadkhin uses to stimulate biologically active points to the scalp to communicate with the hypothalamus, can be likened to a pacemaker. The pacemaker's function is to regulate the beating of the heart, thus restoring its proper rhythm. The tsubo balls have a similar effect on hunger by regulating the “big” clock, or hypothalamus.

Thus, while there have been shown and described and pointed out fundamental novel features of the invention as applied to preferred embodiments thereof, it will be understood that various omissions and substitutions and changes in the form and details of the devices and methods illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. It is the intention, therefore, to be limited only as indicated by the scope of the claims appended hereto.

I claim:

1. A chronocentric method for reducing an individual's weight through hunger control, comprising the steps of:
(a) conducting at least one comprehensive multi-profile diagnostic of a target living organism, to identify at least one of: a target organ, and/or a target organ system, that requires at least one predetermined treatment;
(b) establishing at least one optimal recommended treatment type in response to the at least one predetermined required treatment, for said identified at least one target organ and/or target organ system;
(c) determining a chronosystem context of biological rhythms of said entire target organism, said at least one of a target organ, and/or a target organ system; and
(d) conducting, said at least one optimal recommended treatment, cyclically, on the basis of said target chronosystem context of said target living organism, of at least one of said target organs, and/or of said target organ system.

2. The method of claim 1, wherein said step (d) comprises the step of:
(e) varying at least one of: dosage, frequency, and/or periodicity of said at least one optimal recommended treatment application.

3. The method of claim 1, wherein said step (d) comprises the step of:
(f) varying at least one treatment application parameter, of said at least one optimal recommended treatment application, in accordance with a predefined correlation between said target chronosystem and least one predetermined chonologic factor.
